(19) United States
(12) Patent Application Publication
(10) Pub. No.: US 2026/0202341 A1
(43) Pub. Date: Jul. 16, 2026

(54) OPTICAL WAVEGUIDE SENSOR FOR DETECTING HEAVY METAL IONS AND METHOD OF FABRICATING THE SAME

(71) Applicant: VULCAN PHOTONICS SDN. BHD., Kuala Lumpur (MY)

(72) Inventors: Khong Nee KOO, Ayer Itam (MY); Mei Qun SEAH, Subang Jaya (MY); Farah Hidayah Binti JAMALUDIN, Johor Bahru (MY)

(21) Appl. No.: 19/135,068

(22) PCT Filed: Dec. 2, 2022
(86) PCT No.: PCT/MY2022/050125

§ 371 Date: Jun. 2, 2025

(51) Int. Cl. G01N 21/77; G01N 33/18

(52) U.S. Cl. CPC G01N 21/7703; G01N 33/1813

(57) ABSTRACT

A waveguide optical sensor for detecting heavy metal ions in an aqueous solution and a method for fabricating the waveguide sensor are disclosed. The optical waveguide sensor includes a substrate surface having a uniform thin layer of ligand coupled thereto with a tin catalyst for forming a functionalized substrate surface, where the optical waveguide sensor is configured to be interactable with a light wave at an input of the optical waveguide sensor and an aqueous solution in contact with functionalized substrate surface for determining concentration of heavy metal ions in the aqueous solution, whereby the heavy metal ions in the aqueous solution forms a coordination complex upon contact with a plurality of binding sites available on the functionalized substrate surface, consequently changing the material optical refractive index of the functionalized substrate surface which change corresponds to the concentration of the heavy metal ions.

### FIELD OF INVENTION

The present invention generally relates to detection of heavy metal ions, and more particularly to an optical waveguide sensor and method for fabricating an optical waveguide sensor for detecting heavy metal ions in an aqueous solution.

### BACKGROUND

Heavy metal ions are nonbiodegradable chemicals that constitute a significant threat to human health. Exposure to heavy metal ions such as lead (Pb), cadmium (Cd), mercury (Hg), uranium (U), chromium (Cr), arsenic (As) and others is harmful to human health as it can cause cardiovascular diseases, neurological disorders, cancer and even death. Heavy metal ions released in wastewater through industrial, agricultural, and domestic activities can also cause serious pollution to the environment. Hence, detection of heavy metal ions is crucial for both human health and environmental protection.

Optical thin-film sensors have recently been used for detection of heavy metal ion concentration due to numerous advantages such as compact size, lightweight, inexpensive, fast response time and flexibility for in-situ or remote monitoring. Heavy metal ions can be detected through the thin-film coating of material that is selectively reactive to metal ions resulting in a refractive index change in the sensing region of the optical sensor.

U.S. Pat. No. 7,776,611 B2 relates to functionalized composite materials having selective absorption for specific analytes, methods for the preparation of such materials as films, and optical sensors employing such films for optical detection of analytes. Although the employment of thin-film coating has been disclosed, there is still a difficulty in forming a uniform layer of the thin-film coating in the sensing region of the optical sensor. Non-uniform coating layer often results in non-repeatability and non-reproducibility of the optical sensor.

Furthermore, the sensors and methods currently provided in the art for detecting heavy metal ions typically involve the use of silylating agents with trisubstituted silyl groups to react with the silicon or silica substrate, resulting in agglomeration that decreases surface uniformity. Other than that, the currently available sensors are typically non-tuneable in terms of sensitivity and detection range, complicated and costly to fabricate. Therefore, the present invention aims to provide an improved optical waveguide sensor having a uniform thin-film coating of ligands and method of fabricating the same that can overcome or mitigate some of the aforementioned problems of the prior art.

### SUMMARY

The following presents a simplified summary of the invention in order to provide a basic understanding of some aspects of the invention. This summary is not an extensive overview of the invention. Its sole purpose is to present some concepts of the invention in a simplified form as a prelude to the more detailed description that is presented later. In one aspect, the present invention provides a method for forming an optical waveguide sensor for detecting various types of heavy metal ions in an aqueous solution, the method comprising the steps of treating a substrate surface by cleaning the substrate surface with acetone and isopropanol for enabling coupling of the treated surface with a ligand, wherein the substrate is selected from a group comprising a silica or a silicon substrate; coating the treated substrate surface with the ligand for forming a uniform thin layer of ligand on the substrate surface thereby forming a functionalized substrate surface; and cleaning the functionalized substrate surface with diluted nitric acid thereby forming the optical waveguide sensor.

The method of the present invention can be characterized by performing the coating of the treated substrate surface with the ligand using a tin catalyst, and the ligand is selected from a group comprising carboxylic acid functional group containing organic molecules, amine functional group, or a hydroxyl functional group, whereby the selection of the ligand varies depending on the type of substrate surface and the types of heavy metal ions to be detected, and wherein the optical waveguide sensor is configured to be interactable with a light wave at an input of the optical waveguide sensor and an aqueous solution in contact with functionalized substrate surface for determining concentration of heavy metal ions in the aqueous solution, whereby the heavy metal ions in the aqueous solution forms a coordination complex upon contact with a plurality of binding sites available on the functionalized substrate surface, consequently changing the material optical refractive index of the functionalized substrate surface which change corresponds to the concentration of the heavy metal ions.

Typically, the silicon substrate is further treated by coating a layer of silicon dioxide on the silicon substrate surface, prior to cleaning the substrate surface.

Typically, the treated silicon substrate is coated with the ligand selected from a group comprising carboxylic acid functional group containing organic molecules selected from the group comprising Ethylenediaminetetraacetic acid (EDTA), 4-carboxybenzo-15-crown-5,4-carboxybenzo-18-crown-6, malic acid, maleic acid, oxalic acid, succinic acid, glutaric acid, tannic acid, polyacrylic acid, via esterification, for forming a first functionalized silicon substrate surface or a coated crosslinkable silicon substrate.

In another embodiment, the method further comprises the steps of coating the coated crosslinkable silicon substrate with a further ligand for forming a second functionalized silicon substrate, whereby the further ligand comprises an amine group selected from a group comprising aza 15-crown-5,4-aminobenzo-15-crown 5, or 4-aminobenzo-18-crown-6.

Typically, the amine group is coated to the coated crosslinkable silicon substrate or the double layer crosslinkable silicon substrate via amine conjugation using 1-Ethyl-3-(3-dimethylaminopropyl)-carbodiimide) (EDC) and N-Hydroxysuccinimide (NHS).

In yet another embodiment, the carboxylic acid functional group molecules for forming the coated crosslinkable silicon substrate is selected from a group comprising di- or poly carboxylic acid molecules.

Typically, the di- or poly carboxylic acid molecules is selected from a group comprising malic acid, maleic acid, oxalic acid, succinic acid, glutaric acid, tannic acid or polyacrylic acid.

In a further embodiment, the method further comprises coating the coated crosslinkable silicon substrate with a hydroxyl functional group for forming a double layer crosslinkable silicon substrate, subsequently coating the double layer crosslinkable silicon substrate with the carboxylic acid functional group for forming a third functionalized silicon substrate.

Typically, the hydroxyl functional group is selected from a group comprising di- or poly alcohols.

Typically, the di- or poly alcohols are selected from a group comprising ethylene glycol, propylene glycol, xylitol, erythritol, hydroxy phenol, or polyvinyl alcohol.

Advantageously, the method of the present invention avoids the use of silylating agents to couple selected ligands onto the substrate resulting in a uniform thin film coating of ligand onto the substrate and therefore allowing for repeatability and reproducibility of the optical waveguide sensor. The present invention involves a layer-by-layer reaction whereby the substrate is first coated with carboxylic acid groups and therefore only reacts with the silica present on the treated silicon or silica substrate via the Fischer esterification reaction, which thus eliminates the possibility of agglomeration and improves the surface uniformity.

In an embodiment, the heavy metal ions are detectable by the optical waveguide sensor containing either the first, second, or the third functionalized substrate is selected from the group comprising cadmium and lead ion, which are configured to form the coordination complex upon contact with the first, second, or the third functionalized silicon substrate.

Typically, the esterification or the amine conjugation steps are repeatable for increasing numbers of the plurality of binding sites on the functionalized silicon substrate.

Advantageously, the coating of di- or poly carboxylic acid molecules results in a substrate surface with varying amount of carboxylic acid groups, which provides possibilities for coupling with amine or hydroxyl functional group containing organic molecules resulting in tuneable sensitivity and concentration range of the waveguide sensor, as more binding sites on the sensor allows for more heavy metal ions to bind with the ligands on the functionalized substrate to produce signal change in the sensor.

In another aspect, there is provided an optical waveguide sensor for detecting heavy metal ions in aqueous solution, wherein the optical wave guide sensor is fabricated by the abovementioned methods.

In yet another aspect, there is provided an optical waveguide sensor for detecting heavy metal ions in aqueous solution comprising a substrate surface having a uniform thin layer of ligand coupled thereto for forming a functionalized substrate surface, the substrate being selected from a group comprising a silica or a silicon substrate; characterized in that the substrate surface is coupled with the ligand using a tin catalyst, wherein the ligand is selected from a group comprising carboxylic acid functional group containing organic molecules, amine functional group, or a hydroxyl functional group, whereby the selection of the ligand varies depending on the type of substrate surface and the types of heavy metal ions to be detected, and wherein the optical waveguide sensor is configured to be interactable with a light wave at an input of the optical waveguide sensor and an aqueous solution in contact with functionalized substrate surface for determining concentration of heavy metal ions in the aqueous solution, whereby the heavy metal ions in the aqueous solution forms a coordination complex upon contact with a plurality of binding sites available on the functionalized substrate surface, consequently changing the material optical refractive index of the functionalized substrate surface which change corresponds to the concentration of the heavy metal ions.

In an embodiment, the carboxylic acid functional group is coupled to the substrate through esterification for forming a first functionalized substrate.

In another embodiment, the substrate is coated with a di- or poly carboxylic acid group coupleable to the amine functional group for forming a second functionalized substrate.

In yet another embodiment, the substrate coated with the di- or poly carboxylic acid group is configured to be coupleable to a di- or poly hydroxyl functional group for forming a double coated substrate.

In a further embodiment, the double coated substrate is configured to be couplable to the carboxylic acid functional group for forming a third functionalized substrate.

Advantageously, the sensitivity and detection range of the optical waveguide sensor can be tuned by modifying the crosslinkers and ligands used in forming the functionalized substrate. The additional crosslinkers used for fabricating the third functionalized substrate group provides additional binding sites to the optical waveguide sensor, thereby modifying the sensitivity and detection range of the optical waveguide sensor. The Fisher esterification or amine conjugation can be repeated to add more crosslinkers and more ligands for increasing the number of binding sites. The simple processes used for forming the functionalized substrate may potentially ease mass production of the optical waveguide sensor.

### BRIEF DESCRIPTION OF DRAWINGS

For the purpose of facilitating an understanding of the invention, there is illustrated in the accompanying drawings the preferred embodiments from an inspection of which when considered in connection with the following description, the invention, its construction and operation and many of its advantages would be readily understood and appreciated.

FIG. 1 illustrates the methods of coupling a substrate of an optical waveguide sensor with selected crosslinkers and ligands according to the present invention.

FIG. 2 illustrates the examples of substrates coupled with selected ligands in use for detecting heavy metal ions in an aqueous solution according to the present invention.

FIG. 3 illustrates an example of wavelength shift with respect to material index changes according to the present invention.

FIGS. 4A and 4B illustrate examples of wavelength shift due to increased ions concentration according to the present invention.

FIGS. 5A, 5B, and 5C illustrate examples of the method for fabricating the optical wave guide having the first, second, and third functionalized substrate surface, respectively, according to the present invention.

### DETAILED DESCRIPTION

Exemplary embodiments of the present invention will now be described in detail with reference to the annexed drawings. The following detailed description includes the preferred embodiments of the present invention and should be taken as examples, without limiting the scope of the invention.

The term crosslinkers (or crosslinking reagents) refers to molecules that contain two or more reactive ends capable or chemically attaching to specific functional groups (primary amines, sulfhydryls, etc.) on proteins or other molecules.

The term esterification, in particular, Fischer esterification, refers to an organic reaction employed to convert carboxylic acids in presence of excess alcohol and a strong acid catalyst to form an ester as the final product.

The term ligand refers to an ion or molecule that can donate a pair of electrons to a metal ion to form a coordination complex.

The present invention provides a method for forming an optical waveguide sensor for detecting various types of heavy metal ions contained in an aqueous solution. The optical waveguide sensor mainly comprises of a substrate, coated with a ligand. In order to form the optical waveguide sensor, the substrate needs to be functionalized by coupling the substrate with the ligand by coating the substrate with the ligand via esterification or amine conjugation.

Typically, the substrate is selected from a group comprising silica (SiO₂) or silicon (Si). In order to coat the substrate with the ligand, it is essential to first treat the substrate surface by cleaning the substrate surface with acetone and isopropanol, in order to enable reaction on the substrate surface with the ligand. Additionally, if the silicon substrate is chosen, it is preferred for the silicon substrate to be pre-treated via surface passivation by coating a layer of silicon dioxide, followed by cleaning the pre-treated silicon substrate with acetone and isopropanol.

Next, the treated substrate can be coated with a ligand in presence of a tin catalyst such as dibutyltin dilaurate or tin (II) chloride for forming a uniform thin layer of ligand on the substrate surface, thereby forming the functionalized substrate surface. The coating can be done either by Fisher esterification for ligand selected from a group comprising carboxylic acid functional group selected from a group comprising Ethylenediaminetetraacetic acid (EDTA), 4-carboxybenzo-15-crown-5,4-carboxybenzo-18-crown-6, malic acid, maleic acid, oxalic acid, succinic acid, glutaric acid, tannic acid, polyacrylic acid, or amine conjugation for ligand selected from a group comprising aza 15-crown-5,4-Aminobenzo-15-crown 5, or 4-aminobenzo-18-crown-6.

Finally, the functionalized substrate surface is cleaned with diluted nitric acid to form the functionalized substrate surface that may be used as the optical waveguide sensor. The final cleaning step is performed in order to remove any trace amount of tin catalyst adsorbed on the surface. The functionalized substrate surface, which acts as ligand of the optical waveguide sensor, is configured to be interactable with a light wave at an input of the optical waveguide sensor and the aqueous solution containing heavy metal ions, when the aqueous solution is in contact with functionalized substrate surface. Once the aqueous solution contacts the surface of the functionalized substrate, which contains a plurality of metal ions binding site from the coated ligand, the heavy metal ions in the aqueous solution forms a coordination complex as a reaction. The reaction changes the functionalized substrate surface, which then changes the path of the light wave travelling on the functionalized substrate surface, depending on the concentration of the heavy metal ions available in the aqueous solution. Measurement of the changing optical refractive index of the functionalized substrate surface can then be made, thereby enabling determination of concentration of heavy metal ions in the aqueous solution by measuring the shifting of the travelling path of the light wave.

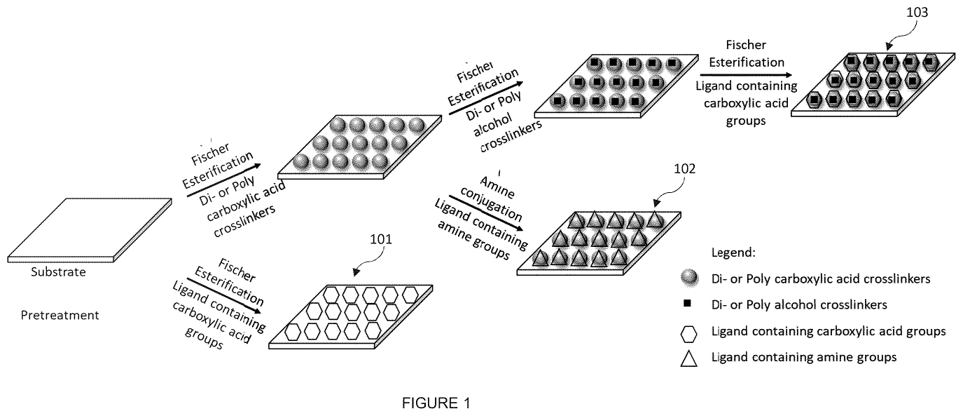

As illustrated in FIG. 1, there are many carboxylic acid containing functional groups that can be used as a ligand, such as Ethylenediaminetetraacetic acid (EDTA), crown ethers, and di- or poly carboxylic acid molecules including malic acid, oxalic acid, succinic acid, glutaric acid, tannic acid and polyacrylic acid. The types of carboxylic acid functional group to be coated on the silicon substrate surface depends on the type of metal ions to be sensed by the optical waveguide sensor. For example, a less ion specific functionalized substrate can be created by coating the substrate with EDTA, which enables sensing of both cadmium and lead ions in an aqueous solution, i.e., more selectivity. For sensing metal ions with less selectivity yet high specificity and sensitivity, the coating on the substrate can be made with crown ethers such as 4-carboxybenzo-15-crown-5 for cadmium ions or aza 15-crown-5 for lead.

The di- or poly carboxylic acid also functions as carboxylic acid molecules crosslinker. Hence, when the carboxylic acid molecules selected to coat the silicon substrate surface are from the group comprising di- or poly carboxylic acid, the coated silicon substrate forms a coated crosslinkable silicon substrate, whereby the coated crosslinkable silicon substrate may be further coated with an amine group by performing amine conjugation to the coated crosslinkable silicon substrate, thereby forming a second type of functionalized substrate surface.

The coated crosslinkable silicon substrate may be reacted with aza 15-crown-5 or 4-aminobenzo-15-crown 5 for forming a lead ion selective sensor or cadmium ion selective sensor respectively, which forms an amide bond via conjugation using 1-Ethyl-3-(3-dimethylaminopropyl)-carbodiimide) (EDC), and N-Hydroxysuccinimide (NHS). The step of crosslinking the substrate with di- or poly carboxylic acid provides for a versatile method of fabricating the sensor as it allows the substrate to be functionalized with ligands other than those of the carboxylic acid groups.

Further, the di- or poly carboxylic acid coated crosslinkable silicon substrate may be reacted with another crosslinkers via Fisher esterification for coating the coated crosslinkable silicon substrate with a hydroxyl functional group, such as di- or poly alcohols selected from a group including ethylene glycol, propylene glycol, xylitol, erythritol, hydroxy phenol, or polyvinyl alcohol, for forming a double layer crosslinkable silicon substrate. The double layer crosslinkable silicon substrate can then be further coated with a ligand by performing Fisher esterification to a carboxylic acid functional group or by performing amine conjugation with an amine group, for forming a third type of functionalized silicon substrate. This potentially increases the number of metal ions binding sites of the ligand coating of the third functionalized silicon substrate. The esterification and the amine conjugation can be repeated multiple times for increasing numbers of the plurality of binding sites on the functionalized silicon substrate, thereby allowing tuning of selectivity and sensitivity of the optical waveguide sensor.

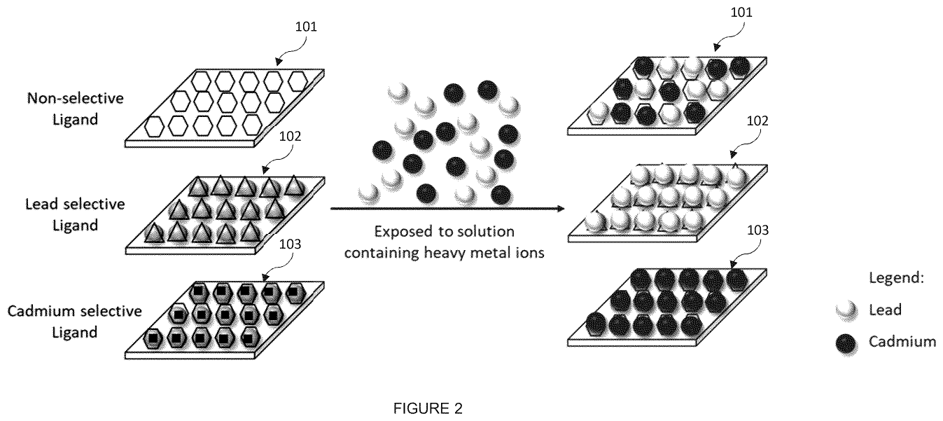

With the above description and as illustrated in FIGS. 1 and 2, it can be understood that the heavy metal ions can be detected by the optical waveguide sensor that employs either the first functionalized silicon substrate 101, the second functionalized silicon substrate 102, or the third functionalized silicon substrate 103, depending on the sensitivity and detection range required by the optical waveguide sensor for detection of heavy metal ions in the aqueous solution, such as cadmium and lead ion. Once the aqueous solution is in contact with the ligands (the first 101, second 102, or third 103 functionalized silicon substrate) of the optical waveguide sensor of the present invention, the heavy metal ions then react with the surface of the ligands and changes the material optical refractive index of the ligand, which changes with respect to absorption kinetics of the targeted metal cations on the ligands. The wavelength of the light wave passing through the ligands will then changes, which can then be measured accordingly. Since different ligands have different absorption kinetics towards the heavy metal ions, the optical waveguide sensor containing the first 101, second 102, and third 103 functionalized silicon substrate can be used in parallel for detecting the heavy metal ions for increasing accuracy in detecting quantity of heavy metal ions present in the aqueous solution.

In accordance with an embodiment of the present invention, there is provided an optical waveguide sensor for detecting heavy metal ions present in aqueous solution, which is prepared and manufactured with the abovementioned method, comprising a substrate coated with a ligand as fabricated with the abovementioned method.

The optical waveguide comprises a substrate surface having a uniform thin layer of ligand coupled thereto for forming a functionalized substrate surface. It is preferred that substrate is selected from a group comprising silica or silicon, with the ligand selected from a group comprising carboxylic acid functional group containing organic molecules, amine functional group, or a hydroxyl functional group, whereby the selection of the ligand varies depending on the type of substrate surface and the types of heavy metal ions to be detected.

Typically, the carboxylic acid functional group is coupled to the substrate by coating the substrate with the carboxylic acid functional group via esterification for forming a first type of functionalized substrate surface 101. Further, the substrate may be coated with a di- or poly carboxylic acid group for forming a coated crosslinkable substrate surface. The di- or poly carboxylic acid group acts as a crosslinker on the substrate surface, which enables coupling of an amine group to the coated crosslinkable substrate surface by coating the amine group on the coated crosslinkable substrate surface via amine conjugation process, consequently forming a second functionalized substrate surface 102.

According to another embodiment, the coated crosslinkable substrate surface is configured to be coupleable with a di- or poly hydroxyl functional group for forming a double coated crosslinkable substrate surface, by coating the coated crosslinkable substrate surface with the di- or poly hydroxyl functional group via Fisher esterification process. The di- or poly hydroxyl functional group acts as another crosslinker, for enabling coupling of the double coated crosslinkable substrate surface with another carboxylic acid functional group or amine group, for forming a third functionalized substrate 103 that acts as a ligand for detecting metal ions available in an aqueous solution.

The crosslinkers can be selected with different amount of carboxylic acid groups and hydroxyl groups to react with the carboxylic acid, hydroxyl or amine groups on the ligands. Hence, the amount of carboxylic acid groups and hydroxyl groups determines the amount of ligands present on the sensor.

The sensitivity and detection range of the sensor can be tuneable by changing the crosslinkers to change the ligand loading to affect the thickness of the coating layer of the substrate surface. The closer the coating layer is to the surface of the waveguide the more sensitive the sensor is to the ions absorbed. Hence, thinner layer with lesser ligands will have a higher sensitivity. However, with a thinner layer, the amount of ions that can be absorbed is lesser, thus the layer will be saturated with a smaller amount of ions which translates to a lower detection range.

The optical waveguide sensor may be connected to a computing device to train an artificial neural network to provide information on the heavy metal ions present in an aqueous solution such as concentrations of the heavy metal ions. This can be done through a deconvolution algorithm stored in an application, which is installed to the computing medium and connected to the optical waveguide sensor. The functionalized substrate surface may be integrated with the optical waveguide sensor, such that the resonant wavelength of the laser passed across the functionalized surface shifts accordingly to the change of material index due to the absorption of the ions in the functionalized substrate surface, therefore the concentration of the ions in the aqueous solution can be measured and quantified by detecting the shift in resonant wavelength curves against time curves. The same technique may be used to train artificial neural network for deriving the concentration of the ions.

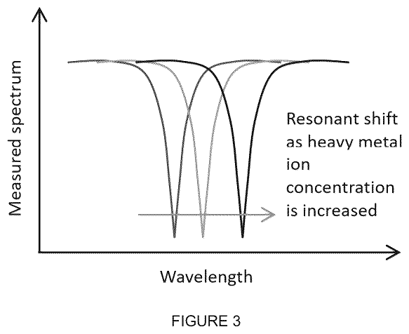

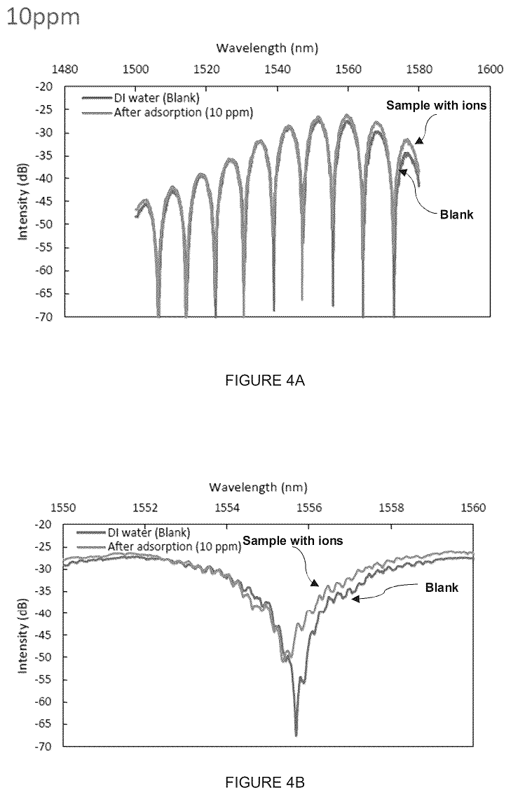

An example of wavelength shifts of the optical waveguide sensor integrated with the functionalized substrate surface due to the change of material index is shown in FIG. 3. Accordingly, as the functionalized substrate surface absorbs the ion, the change in the material index causes a shift in the wavelength, which facilitates in measuring or quantifying the ion concentration in the aqueous solution. This is shown in FIGS. 4A and 4B, in which the wavelength shifts as the functionalized substrate surface absorbs more ions from an aqueous solution. The presence of ions can be detected within a predetermined exposure time, for example within 20 to 120 seconds. It has been found that the change or wavelength shift does not substantially change further after 120 seconds.

The embodiment of the present invention will now be described with the following examples.

The examples provided herein are intended to illustrate the different aspects and embodiments of the invention. The examples are not intended in any way to limit the disclosed invention, which is limited only by the appended claims.

### Example 1

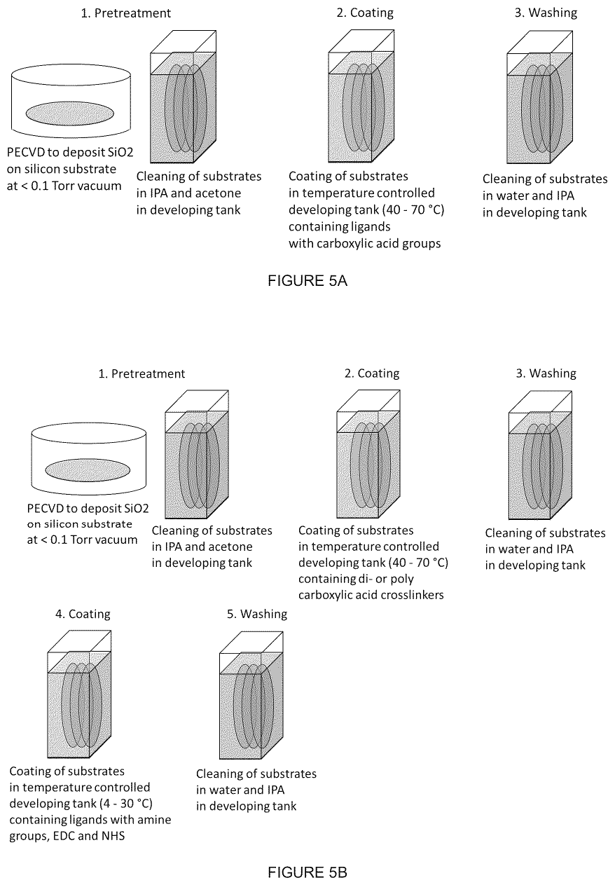

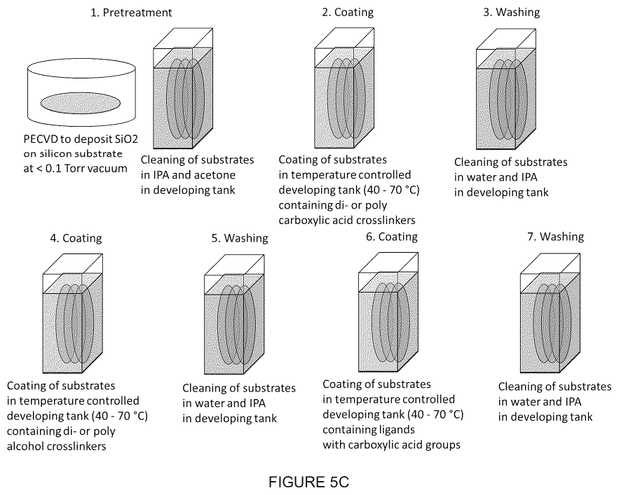

The method of fabricating the sensor involves plasma-enhanced chemical vapor deposition (PECVD) if the substrate selected is silicon at a room temperature and at vacuum pressure of less than 0.1 Torr. Subsequent steps are carried out in atmospheric pressure. For silica and silica coated silicon substrates, the substrates can be cleaned in developing tanks containing IPA and acetone. After which, coating of the ligand containing carboxylic acid groups can be done by submerging the substrates in a temperature controlled (40-70° C.) developing tank containing the ligand with carboxylic acid groups. Finally, the substrates can be washed using water and IPA in separate developing tanks. The above process flow is as illustrated in FIG. 5A.

### Example 2

The method of fabricating the sensor involves plasma-enhanced chemical vapor deposition (PECVD) if the substrate selected is silicon at a room temperature and at vacuum pressure of less than 0.1 Torr. Subsequent steps are carried out in atmospheric pressure. For silica and silica coated silicon substrates, the substrates can be cleaned in developing tanks containing IPA and acetone. After which, coating of the di- or poly-carboxylic acid can be done by submerging the substrates in a temperature controlled (40-70° C.) developing tank containing the di- or poly-carboxylic acid. This step is then followed by a washing step to wash the substrates with water and IPA in separate developing tanks. Following which, the substrates are then coated with ligands with amine groups, by submerging the substrates in a temperature controlled (4-30° C.) developing tank containing the ligand with amine groups, EDC and NHS. Finally, the substrates can be washed using water and IPA in separate developing tanks. The above process flow is as illustrated in FIG. 5B.

### Example 3

The method of fabricating the sensor involves plasma-enhanced chemical vapor deposition (PECVD) if the substrate selected is silicon at a room temperature and at vacuum pressure of less than 0.1 Torr. Subsequent steps are carried out in atmospheric pressure. For silica and silica coated silicon substrates, it can be cleaned in developing tanks containing IPA and acetone. After which, coating of the di- or poly-carboxylic acid can be done by submerging the substrates in a temperature controlled (40-70° C.) developing tank containing the di- or poly-carboxylic acid. This step is then followed by a washing step to wash the substrates with water and IPA in separate developing tanks. Following which, di- or poly-alcohols can be coated by submerging the substrates in a temperature controlled (40-70° C.) developing tank containing the di- or poly-alcohols. This step is then followed by a washing step to wash the substrates with water and IPA in separate developing tanks. After which, the substrates can be coated with the ligand containing carboxylic acid groups by submerging the substrates in a temperature controlled (40-70° C.) developing tank containing the ligand with carboxylic acid groups. Finally, the substrates can be washed using water and IPA in separate developing tanks. The above process flow is as illustrated in FIG. 5C.

### Example 4

Table 1 shows a list of examples of possible configurations of the first (101), second (102), and third (103) functionalized substrates together with the crosslinkers and/or ligands used for detecting the heavy metal ion of interest according to an embodiment of the present invention.

TABLE 1
Types of substrates, crosslinkers, and/or ligands
used for detecting the specific heavy metal ions of interest.

| | | Di- or poly- | | Ligands | Ligands | |
|---|---|---|---|---|---|---|
| | | carboxylic | Di- or poly- | containing | containing | Ions |
| | | acid | alcohol | amine | carboxylic | of |
| | Substrates | crosslinkers | crosslinkers | groups | acid groups | interest |
| 101 | Si/SiO₂ | — | — | — | 4- | Cd |
| | | | | | carboxybenzo- | |
| | | | | | 15-crown-5 | |
| | | — | — | — | EDTA | Cd, Pb |
| | | | | | | and Cr |
| | | — | — | — | Iron and | As |
| | | | | | 4- | |
| | | | | | carboxybenzo- | |
| | | | | | 18-crown-6 | |
| | | | | | complex | |
| | | — | — | — | 4- | Hg |
| | | | | | carboxybenzo- | and U |
| | | | | | 18-crown-6 | |
| | | — | — | — | Iron and di- | As |
| | | | | | carboxy | |
| | | | | | phenanthroline | |
| | | | | | complex | |
| 102 | Si/SiO₂ | Malic acid | — | 4- | — | Cd |
| | | | | Aminobenzo- | | |
| | | | | 15-crown | | |
| | | | | 5 | | |
| | | Maleic acid | — | aza 15- | — | Pb |
| | | | | crown-5 | | |
| | | Succinic | — | Iron and | — | As |
| | | acid | | 4- | | |
| | | | | Aminobenzo- | | |
| | | | | 18- | | |
| | | | | crown-6 | | |
| | | | | complex | | |
| | | Glutaric acid | — | 4- | — | Hg |
| | | | | aminobenzo- | | and U |
| | | | | 18- | | |
| | | | | crown-6 | | |
| | | Oxalic acid | — | — | — | — |
| | | Tannic acid | — | — | — | — |
| | | Poly acrylic | — | — | — | — |
| | | acid (MW: | | | | |
| | | 0.45 mil-5 mil) | | | | |
| 103 | Si/SiO₂ | Malic acid | Ethylene | — | 4- | Cd |
| | | | glycol | | carboxybenzo- | |
| | | | | | 15-crown-5 | |
| | | Maleic acid | Propylene | — | EDTA | Cd, Pb |
| | | | glycol | | | and Cr |
| | | Succinic | xylitol | — | Iron and | As |
| | | acid | | | 4- | |
| | | | | | carboxybenzo- | |
| | | | | | 18-crown-6 | |
| | | | | | complex | |
| | | Glutaric acid | Erythritol | — | 4- | Hg |
| | | | | | carboxybenzo- | and U |
| | | | | | 18-crown-6 | |
| | | Oxalic acid | Hydroxy | — | Iron and di- | As |
| | | | phenol | | carboxy | |
| | | | | | phenanthroline | |
| | | | | | complex | |
| | | Tannic acid | polyvinyl | — | — | — |
| | | | alcohol | | | |
| | | | (MW: 9k - | | | |
| | | | 186k) | | | |
| | | Poly acrylic | — | — | — | — |
| | | acid (MW: | | | | |
| | | 0.45 mil-5 mil) | | | | |

The above-described embodiments of a method to fabricate the optical waveguide sensor only utilizes non-toxic solvents such as water and ethanol. Therefore, the above-described fabrication process may potentially improve sustainability in producing the optical sensor, in comparison to the optical waveguide sensor fabricated with methods of the prior art. Further, the sensitivity of heavy metal ions sensing of the optical waveguide sensor produced by the above-described method may be tuneable, thereby improving portability and simplicity with sensing heavy metal ions in water in comparison to inductively coupled plasma atomic emission spectroscopy method, ICP-OES. Further, the method of the present invention improves scalability for mass production of the optical waveguide sensor of the present invention. This is made possible with the use of only two sets of equipment, mainly a PECVD chamber and a developer tank, resulting to a highly versatile method which could be easily performed in series and modified according to the required specifications of the sensor.

Next, the method of the present invention may potentially ease modification of the optical waveguide sensors to cater for different heavy metal ions. The crosslinkers and ligands can be changed along with the temperature of 40-70° C. for Fischer esterification and 4-30° C. for amine conjugation) with the duration of 30-60 min to coat different thin films for different heavy metal ions. Also, the method of the present invention eliminates the use of silylating agents which are moisture and pH sensitive and requires more stringent process control and anhydrous solvent. For water soluble reactants, water can be used as the reaction medium which makes the process more environmentally friendly and less toxic and flammable. Further, the optical waveguide sensor of the present invention may utilize the unique absorption kinetics of different heavy metal ions on the different ligands to give a quantitative result of the heavy metal ions present in the aqueous solution. This is achievable through measurement of resonant wavelength shift of a laser passed across the functionalized substrate of the optical waveguide sensor over time, which could then be deconvoluted via the computing medium, which can be a personal computer or a smart device such as smartphones.

While the invention has been described as required in terms in preferred embodiments and specific operating ranges and conditions, those skilled in the art will appreciate that the invention described herein is susceptible to variations and modifications other than those specifically described.

1. A method for forming an optical waveguide sensor for detecting various types of heavy metal ions in an aqueous solution, the method comprising the steps of:
treating a substrate surface by cleaning the substrate surface with acetone and isopropanol for enabling coupling of the treated surface with a ligand, the substrate being selected from a group comprising a silica or a silicon substrate;
coating the treated substrate surface with the ligand for forming a uniform thin layer of ligand on the substrate surface thereby forming a functionalized substrate surface; and
cleaning the functionalized substrate surface with diluted nitric acid thereby forming the optical waveguide sensor,
wherein the ligand is selected from a group comprising carboxylic acid functional group containing organic molecules, amine functional group, or a hydroxyl functional group, whereby the selection of the ligand varies depending on the type of substrate surface and the types of heavy metal ions to be detected; and
the coating of the treated substrate surface with the ligand is performed with a tin catalyst by Fisher esterification when the ligand comprises either a carboxylic acid functional group containing organic molecules or a hydroxyl functional group, or by amine conjugation when the ligand comprises an amine functional group, and
wherein the optical waveguide sensor is configured to be interactable with a light wave at an input of the optical waveguide sensor and an aqueous solution in contact with functionalized substrate surface for determining concentration of heavy metal ions in the aqueous solution, whereby the heavy metal ions in the aqueous solution forms a coordination complex upon contact with a plurality of binding sites available on the functionalized substrate surface, consequently changing a material optical refractive index of the functionalized substrate surface which change corresponds to the concentration of the heavy metal ions.

2. The method according to claim 1, wherein the silicon substrate is further treated by coating a layer of silicon dioxide on the silicon substrate surface, prior to cleaning the substrate surface.

3. The method according to claim 1, wherein the treated silicon substrate is coated with the ligand comprising a carboxylic acid functional group containing organic molecules selected from a group comprising Ethylenediaminetetraacetic acid (EDTA), 4-carboxybenzo-15-crown-5,4-carboxybenzo-18-crown-6, malic acid, maleic acid, oxalic acid, succinic acid, glutaric acid, tannic acid polyacrylic acid, via esterification, for forming a first functionalized silicon substrate surface or a coated crosslinkable silicon substrate.

4. The method according to claim 3, further comprising the steps of coating the coated crosslinkable silicon substrate with a further ligand for forming a second functionalized silicon substrate, whereby the further ligand comprises an amine group selected from a group comprising aza 15-crown-5,4-Aminobenzo-15-crown 5, or 4-aminobenzo-18-crown-6.

5. The method according to claim 4, wherein the amine group is coated to the coated crosslinkable silicon substrate or the double layer crosslinkable silicon substrate via amine conjugation using 1-Ethyl-3-(3-dimethylaminopropyl)-carbodiimide) (EDC) and N-Hydroxysuccinimide (NHS).

6. The method according to claim 3, wherein the carboxylic acid functional group molecules for forming the coated crosslinkable silicon substrate is selected from a group comprising di- or poly carboxylic acid molecules.

7. The method according to claim 6, wherein the di- or poly carboxylic acid molecules is selected from a group comprising malic acid, maleic acid, oxalic acid, succinic acid, glutaric tannic acid, or polyacrylic acid.

8. The method according to claim 6, further comprising coating the coated crosslinkable silicon substrate with a hydroxyl functional group for forming a double layer crosslinkable silicon substrate, subsequently coating the double layer crosslinkable silicon substrate with the carboxylic acid functional group for forming a third functionalized silicon substrate.

9. The method according to claim 8, wherein the hydroxyl functional group is selected from a group comprising di- or poly alcohols.

10. The method according to claim 9, wherein the di- or poly alcohols are selected from a group comprising ethylene glycol, propylene glycol, xylitol, erythritol, hydroxy phenol, or polyvinyl alcohol.

11. The method according to claim 1, wherein the heavy metal ions are detectable by the optical waveguide sensor containing either the first, second, or the third functionalized substrate is selected from the group comprising cadmium and lead ion, which are configured to form the coordination complex upon contact with the first, second, or the third functionalized silicon substrate.

12. The method according to claim 1, wherein the esterification or the amine conjugation steps are repeatable for increasing numbers of the plurality of binding sites on the functionalized silicon substrate.

13. An optical waveguide sensor for detecting heavy metal ions in aqueous solution, wherein the optical wave guide sensor is fabricated by the steps of claim 11.

14. An optical waveguide sensor for detecting heavy metal ions in aqueous solution comprising:
a substrate surface having a uniform thin layer of ligand coupled thereto for forming a functionalized substrate surface, the substrate being selected from a group comprising a silica or a silicon substrate;
wherein the ligand is selected from a group comprising carboxylic acid functional group containing organic molecules, amine functional group, or a hydroxyl functional group, whereby the selection of the ligand varies depending on the type of substrate surface and the types of heavy metal ions to be detected; and
the substrate surface is coupled with the ligand using a tin catalyst by Fisher esterification when the ligand is selected from a group comprising carboxylic acid functional group containing organic molecules and hydroxyl functional group, or by amine conjugation when the ligand is selected from a group comprising amine functional group, and
wherein the optical waveguide sensor is configured to be interactable with a light wave at an input of the optical waveguide sensor and an aqueous solution in contact with functionalized substrate surface for determining concentration of heavy metal ions in the aqueous solution, whereby the heavy metal ions in the aqueous solution forms a coordination complex upon contact with a plurality of binding sites available on the functionalized substrate surface, consequently changing a material optical refractive index of the functionalized substrate surface which change corresponds to the concentration of the heavy metal ions.

15. The sensor according to claim 14, wherein the carboxylic acid functional group is coupled to the substrate through esterification for forming a first functionalized substrate.

16. The sensor according to claim 14, wherein the substrate is coated with a di- or poly carboxylic acid group coupleable to the amine functional group for forming a second functionalized substrate.

17. The sensor according to claim 16, wherein the substrate coated with the di- or poly carboxylic acid group is configured to be coupleable to a di- or poly hydroxyl functional group for forming a double coated substrate.

18. The sensor according to claim 17, wherein the double coated substrate is configured to be couplable to the carboxylic acid functional group for forming a third functionalized substrate.
